(19) United States
(12) Patent Application Publication
(10) Pub. No.: US 2014/0046208 A1
(43) Pub. Date: Feb. 13, 2014

(54) COMPRESSIVE SAMPLING OF PHYSIOLOGICAL SIGNALS USING TIME-FREQUENCY DICTIONARIES BASED ON MODULATED DISCRETE PROLATE SPHEROIDAL SEQUENCES

(71) Applicant: UNIVERSITY OF PITTSBURGH-OF THE COMMONWEALTH SYSTEM OF HIGHER EDUCATION, Pittsburgh, PA (US)

(72) Inventors: Ervin Sejdic, Pittsburgh, PA (US); Luis F. Chaparro, Monroeville, PA (US)

(73) Assignee: UNIVERSITY OF PITTSBURGH-OF THE COMMONWEALTH SYSTEM OF HIGHER EDUCATION, Pittsburgh, PA (US)

(21) Appl. No.: 13/961,916

(22) Filed: Aug. 8, 2013

Related U.S. Application Data
(60) Provisional application No. 61/681,427, filed on Aug. 9, 2012.

(51) Int. Cl. A61B 5/00

(52) U.S. Cl. CPC A61B 5/7232
USPC 600/528; 600/300; 600/586

(57) ABSTRACT

A method of sampling and reconstructing an original physiological signal obtained from a subject includes acquiring a number of samples of the original physiological signal, and generating a reconstructed physiological signal using the samples and a time-frequency dictionary, the time-frequency dictionary having bases which are modulated discrete prolate spheroidal sequences.

This application claims priority under 35 U.S.C. §119(e) from U.S. provisional patent application No. 61/681,427, entitled “Compressive Sampling Of Biomedical Signals Using Time-Frequency Dictionaries Based On Modulated Discrete Prolate Spheroidal Sequences” and filed on Aug. 9, 2012, the contents of which are incorporated herein by reference.

### BACKGROUND OF THE INVENTION

1. Field of the Invention

The present invention pertains to the sampling of physiological signals from a subject, such as, without limitation, signals representing physiological functions like swallowing or heart function, and in particular, to systems and methods for sampling and reconstructing an original physiological signal obtained from a subject using time-frequency dictionaries based on modulated discrete prolate spheroidal sequences.

2. Description of the Related Art

Swallowing (deglutition) is a complex process of transporting food or liquid from the mouth to the stomach consisting of four phases: oral preparatory, oral, pharyngeal, and esophageal. Dysphagic patients (i.e., patients suffering from swallowing difficulty) usually deviate from the well-defined pattern of healthy swallowing. Dysphagia frequently develops in stroke patients, head injured patients, and patients with other with paralyzing neurological diseases. Patients with dysphagia are prone to choking and aspiration (the entry of material into the airway below the true vocal folds). Aspiration and dysphagia may lead to serious health sequalae, including malnutrition and dehydration, degradation in psychosocial well-being, aspiration pneumonia, and even death.

The videofluoroscopic swallowing study (VFSS) is used widely in today's dysphagia management and represents the gold standard for dysphagia assessment. However, VFSS requires expensive X-ray equipment as well as expertise from speech-language pathologists and radiologists. Hence, only a limited number of institutions can offer VFSS and the procedure has been associated with long waiting lists.

In addition, day-to-day monitoring of dysphagia is crucial due to the fact that the severity of dysphagia can fluctuate over time, and VFSS, regardless of its availability, is not suitable for such day-to-day monitoring. Thus, other methods must be used for day-to-day monitoring of dysphagia.

Cervical auscultation is a promising non-invasive tool for the assessment of swallowing disorders, including day-to-day monitoring of dysphagia. Cervical auscultation involves the examination of swallowing signals acquired via a stethoscope or some other acoustic and/or vibration sensor during deglutition. Swallowing accelerometry is one such approach and employs an accelerometer as a sensor during cervical auscultation. Swallowing accelerometry has been used to detect aspiration in several studies, which have described a shared pattern among healthy swallow signals, and verified that this pattern is either absent, delayed or aberrant in dysphasic swallow signals.

These previous studies have used single-axis accelerometers to monitor only vibrations propagated in the anterior-posterior direction at the cervical region. Proper hyolaryngeal movement with precise timing during bolus transit is vital for airway protection in swallowing. Since the motion of the hyolaryngeal structure during swallowing occurs in both the anterior-posterior (A-P) and the superior-inferior (S-I) directions, the employment of dual-axis accelerometry seems to be well motivated. A correlation has been reported between the extent of laryngeal elevation and the magnitude of the A-P swallowing accelerometry signal, and thus it has been hypothesized that vibrations in the S-I axis also capture useful information about laryngeal elevation. From a physiological standpoint, the S-I axis appears to be as worthy of investigation as the A-P axis because the maximum excursion of the hyolaryngeal structure during swallowing is of similar magnitude in both the anterior and superior directions. Recent studies have indeed confirmed that dual-axis accelerometers yield more information and enhanced analysis capabilities, and thus appear to be valuable tools for use in assessment of swallowing disorders, including day-to-day monitoring of dysphagia.

In addition, cardiovascular disease remains the leading cause of death worldwide despite numerous advances in monitoring and early detection of the diseases. Fortunately, clinical experience has shown that heart sounds can be an effective tool to noninvasively diagnose some of the heart failures, since they provide clinicians with valuable diagnostic and prognostic information concerning the heart valves and hemodynamics. Heart auscultation is an important technique allowing the detection of abnormal heart behavior before it can be detected using other techniques such as an ECG.

However, continuous monitoring of physiological functions such as, without limitation) swallowing or heart function (heart sounds) as just described, among others, can pose severe constraints on data acquisition and processing systems. This is especially true where the continuous monitoring involves the capture of a very large amount of data, as would be the case if dual-axis accelerometry were to be used for cervical auscultation in day-to-day monitoring of dysphagia. Even sampling physiological signals at relatively low rates (e.g., 250 Hz) will result in close to a million samples in the first hour of monitoring.

Similar computational burdens are present in telemedicine, and in recent years a number of efforts have been made to deal with this problem. One such effort involves compressing the acquired signals immediately upon sampling using various schemas. Other efforts involve rethinking the way the data is acquired, such as be employing what is known as compressive sensing (CS) (also sometimes referred to as compressed sensing). CS is a signal processing technique for efficiently acquiring and reconstructing a signal by finding solutions to underdetermined linear systems. CS takes advantage of the signal's sparseness or compressibility in some domain, allowing the entire signal to be determined from relatively few measurements.

The idea of CS has gained considerable attention in recent years. The main idea behind CS is to diminish the number of steps involved when acquiring data by combining sampling and compression into a single step. More specifically, CS enables one to acquire the data at sub-Nyquist rates, and recover it accurately from such sparse samples.

Traditional (non-CS) signal processing approaches for sensing and processing of information have relied on the Shannon sampling theorem, which states that a band limited signal x(t) can be reconstructed from uniform samples {x(kTs)} as follows:

$$x ( t ) = {\sum }_{k} x ( {kT}_{s} ) \frac{sin ( {Ω}_{max} ( t - {kT}_{s} ) / π )}{{Ω}_{max} ( t - {kT}_{s} ) / π} ,$$

where Ts is the sampling period and Ωₘₐₓ represents the maximum frequency present in the signal. In other words, the Shannon sampling theorem states that in order to ensure accurate representation and reconstruction of a signal with Ωₘₐₓ, the signal should sampled at least at 2Ωₘₐₓ samples per second (i.e., the Nyquist rate). However, a number of recent publications, including Senay et al., “Reconstruction of non-uniformly sampled time-limited signals using prolate spheroidal wave functions,” Signal Processing, vol. 89, no. 12, pp. 2585-2595, December 2009 and H. Mamaghanian et al., “Compressed sensing for real-time energy-efficient ECG compression on wireless body sensor nodes,” IEEE Transactions on Biomedical Engineering, vol. 58, no. 9, pp. 2456-2466, September 2011, have challenged this approach for a number of reasons. First, by using the Shannon sampling theorem, bases of infinite support are relied upon, while in general signal samples are reconstructed in the finite domain. Second, large bandwidth values can severely constrain sampling architectures. Third, even when signals with relatively low bandwidth values, such as swallowing accelerometry signals, are considered, continuous monitoring of swallowing function can produce a large number of redundant samples, which severely constrains processing efforts.

As described in D. L. Donoho, “Compressed sensing,” IEEE Transactions on Information Theory, vol. 52, no. 4, pp. 1289-1306, April 2006, W. Dai et al., “Subspace pursuit for compressive sensing signal reconstruction,” IEEE Transaction on Information Theory, vol. 55, no. 5, pp. 2230-2249, May 2009, and K.-K. Poh et al., “Compressive sampling of EEG signals with finite rate of innovation,” EURASIP Journal on Advances in Signal Processing, vol. 2010, p. 12 pages, 2010, employing CS may resolve some of the aforementioned issues. CS is a method closely related to transform coding, since a transform code converts input signals, embedded in a high-dimensional space, into signals that lie in a space of significantly smaller dimensions (e.g., wavelet and Fourier transforms). CS approaches are particularly suited for K-sparse signals, i.e., signals that can be represented by significant K coefficients over an N-dimensional basis. Encoding of a K-sparse, discrete-time signal of dimension N is accomplished by computing a measurement vector y that consists of M<<N linear projections of the vector x. This can be compactly described as follows:

y=Φx,

where φ represents an M×N matrix and is often referred to as the sensing matrix. A natural formulation of the recovery problem is within a norm minimization framework, which seeks a solution to the problem

min∥x∥₀ subject to ∥y−Φx∥₂<η

where η is the expected noise of measurements, ∥x∥₀ counts the number of nonzero entries of x and ∥∥₂ is the Euclidian norm. Unfortunately, the above minimization is not suitable for many applications as it is NP-hard.

In short, as just described, monitoring physiological functions such as swallowing and heart sounds can be computationally intensive. In other words, monitoring physiological functions such as swallowing and heart sounds can produce a vast amount of data samples which must be stored and processed. It will be understood that this monitoring (e.g., remote monitoring) can be extremely expensive and present computational limitations which sometimes inhibit capture and accurate processing of the data. There is thus a need in the art for an efficient and accurate mechanism to enable this type of monitoring. In particular, what is needed is a CS-based solution that is suitable for sampling and reconstructing physiological signals from a subject, such as, without limitation, signals representing physiological functions like swallowing or heart function.

### SUMMARY OF THE INVENTION

The innovation disclosed and claimed herein, in one aspect thereof, comprises systems and methods that facilitate monitoring physiological functions such as, without limitation, swallowing and heart sounds. As stated above, these types of functions often generate large volumes of samples to be stored and processed, which can introduce computational constraints, especially if remote monitoring is desired. Accordingly, in aspects, the subject innovation discloses a compressive sensing (CS) algorithm to alleviate some of these issues while acquiring, for example and without limitation, dual-axis swallowing accelerometry signals or heart sound signals. The CS approach describe herein uses a time-frequency dictionary where the members are modulated discrete prolate spheroidal sequences (MDPSS). These waveforms are obtained by modulation and variation of discrete prolate spheroidal sequences (DPSS) in order to reflect the time-varying nature of certain physiological signals, such as swallowing accelerometry signals or heart sound signals. While the modulated bases permit one to represent the signal behavior accurately, in the exemplary embodiment the matching pursuit algorithm is adopted to iteratively decompose the signals into an expansion of the dictionary bases.

To test the accuracy of the scheme of the present invention, the present inventors carried out several numerical experiments with synthetic test signals, dual-axis swallowing accelerometry signals and heart sounds. In all cases, the CS approach based on the MDPSS yields very accurate representations. Specifically, the innovation illustrates that dual-axis swallowing accelerometry signals and heart sounds can be accurately reconstructed even when the sampling rate is reduced to half of the Nyquist rate. The results clearly indicate that the approach of the present invention is adequate for compressive sensing of physiological signals such as, without limitation, swallowing accelerometry signals and heart sounds.

In one embodiment, method of sampling and reconstructing an original physiological signal obtained from a subject is provided that includes acquiring a number of samples of the original physiological signal, and generating a reconstructed physiological signal using the samples and a time-frequency dictionary, the time-frequency dictionary having bases which are modulated discrete prolate spheroidal sequences.

In another embodiment, a system for sampling and reconstructing an original physiological signal obtained from a subject is provided that includes an output device and a computing device having a processor apparatus structured and configured to receive a number of samples of the original physiological signal, generate a reconstructed physiological signal using the samples and a time-frequency dictionary, the time-frequency dictionary having bases which are modulated discrete prolate spheroidal sequences, and cause the reconstructed physiological signal to be output on the output device.

In still another embodiment, a system that facilitates the monitoring of physiological function is provided that includes a sampling component that employs compressive sensing of biomedical signals associated with the physiological function, and a dictionary component that employs time-frequency dictionaries based upon modulated discrete prolate spheroidal sequences (DPSS) to process the compressive sensing.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIGS. 1A-1D illustrate alternative methods for partitioning the bandwidth of a DPSS;

FIG. 2 is a flowchart illustrating a method of sampling and reconstructing an original physiological signal obtained from a subject according to the exemplary embodiment of the present invention;

FIG. 3 is a block diagram of a system for day-to-day monitoring of swallowing disorders in which the method of the present invention (e.g., FIG. 2) may be implemented according to one particular, non-limiting exemplary embodiment;

FIG. 4 is a block diagram of a computing device forming a part of the system of FIG. 3 and FIG. 10 according to one exemplary embodiment;

FIGS. 5A-5D, 6A-6D, 7A-7D, 8A-8F, 9A-9F and 11A-11F shows the results of various experiments conducted by the present inventors to demonstrate the effectiveness of the method of the present invention; and

FIG. 10 is a block diagram of a system for monitoring heart sounds in which the method of the present invention (e.g., FIG. 2) may be implemented according to another particular, non-limiting exemplary embodiment.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

As used herein, the term “time-frequency dictionary” means a set of orthogonal or non-orthogonal basis functions covering the time-frequency plane. The time-frequency dictionary can also be composed of orthonormal or non-orthonormal basis functions covering the time-frequency plane.

As used herein, the term “matching pursuit algorithm” means a type of numerical technique which involves finding the “best matching” projections of multidimensional data onto an over-complete dictionary D.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

The present invention will now be described, for purposes of explanation, in connection with numerous specific details in order to provide a thorough understanding of the subject invention. It may be evident, however, that the present invention can be practiced without these specific details. For example, while techniques are employed to monitor specific physiological functions, it is to be understood that deviations can exist while remaining within the spirit and scope of the present invention. More particularly, where swallowing and heart sounds are described in detail, most any physiologic function can be monitored without departing from the spirit and scope of this innovation. These alternatives are to be included within the specification herein.

As used in this application, the terms “component” and “system” are intended to refer to a computer related entity, either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers. While certain ways of displaying information to users are shown and described with respect to certain figures or graphs as screenshots, those skilled in the relevant art will recognize that various other alternatives can be employed. The terms “screen,” “web page,” and “page” are generally used interchangeably herein. The pages or screens are stored and/or transmitted as display descriptions, as graphical user interfaces, or by other methods of depicting information on a screen (whether personal computer, PDA, mobile telephone, or other suitable device, for example) where the layout and information or content to be displayed on the page is stored in memory, database, or another storage facility.

As described in detail herein, the present invention provides an approach for CS of physiological signals, such as, without limitation, swallowing accelerometry signals and heart sound signals, that is based on a time-frequency dictionary. In particular, the members of the dictionary are recently proposed modulated discrete spheroidal sequences (MDPSS). The bases within the time-frequency dictionary are obtained by modulation and variation of the bandwidth of discrete prolate spheroidal sequences (DPSS) to reflect the varying time-frequency nature of many physiological signals, including swallowing accelerometry signals and heart sound signals, among others.

Given the CS framework, the immediate question is how to define the sensing matrix Φ, that is the bases used in the recovery/reconstruction of the original physiological signal. Most commonly used sensing matrices are random matrices with independent identically distributed (i.i.d.) entries formed by sampling either a Gaussian distribution or a symmetric Bernoulli distribution. Previous work has shown that these matrices can recover the signal with high probability. However, when dealing with physiological (biomedical) signals, it is desirable to recover the signals as precisely as possible (i.e., with a very small error). Therefore, as described in greater detail herein, the present invention employs a time-frequency dictionary (also known as frames) that is based on modulated discrete prolate spheroidal sequences (MDPSS).

To understand MDPSS, a general description of discrete prolate spheroidal sequences (DPSS) is helpful. Given N such that n=0, 1, . . . , N−1 and the normalized half-bandwidth, W, such that 0<W<0.5, the kth DPSS, vₖ (n, N, W), is defined as the real solution to the system of equations shown below:

$${\sum }_{m = 0}^{N - 1} \frac{sin [ 2 π W ( n - m ) ]}{π ( n - m )} {v}_{k} ( m , N , W ) = {λ}_{k} ( N , W ) {v}_{k} ( n , N , W ) k = 0 , 1 , … , N - 1 ,$$

with λₖ (N, W) being the ordered non-zero eigenvalues of the above system of equations as shown below:

λ₀(N,W)>λ₁(N,W), . . . , λ_{N-1}(N,W)>0.

It has been shown that behavior of these eigenvalues for fixed k and large N is given by:

$$1 - {λ}_{k} ( N , W ) \sim \frac{\sqrt{π}}{k !} {2 }^{\frac{t 4 k + 9}{4}} {{α}^{\frac{2 k + t}{4}} [ 2 - α ]}^{- ( k + 0.5 )} {N}^{k + 0.5} {e}^{- γ N} , \\ where$$

$$α = 1 - cos ( 2 π W )$$

$$γ = log [ 1 + \frac{2 \sqrt{( α )}}{\sqrt{2} - \sqrt{α}} ] .$$

The first 2NW eigenvalues are very close to 1 while the rest rapidly decay to zero. Interestingly enough, it has been observed that these quantities are also the eigenvalues of N×N matrix C (m, n), where the elements of such a matrix are:

$$C ( m , n ) = \frac{sin [ 2 π W ( n - m ) ]}{π ( n - m )} \qquad m , n = 0 , 1 , … , N - 1 ,$$

and the vector obtained by time-limiting the DPSS, vk (n, N, W), is an eigenvector of C (m, n). The DPSS are doubly orthogonal, that is, they are orthogonal on the infinite set {−∞, . . . , ∞} and orthonormal on the finite set {0, 1, . . . , N−1}, that is,

$${\sum }_{- ∞}^{∞} {v}_{i} ( n , N , W ) {v}_{j} ( n , N , W ) = {λ}_{i} {δ}_{ij}$$

$${\sum }_{n = 0}^{N - 1} {v}_{i} ( n , N , W ) {v}_{j} ( n , N , W ) = {δ}_{ij}$$

where i, j=0, 1, . . . , N−1. The sequences also obey symmetry laws as follows:

vₖ(n,N,W)=(−1)^{k}vₖ(N−1−n,N,W)

vₖ(n,N,W)=(−1)^{k}v_{N-1-k}(N−1−n,N,1/2−W)

where n=0, ±1, ±2, . . . and k=0, 1, . . . , N−1.

If these DPSS are used for signal representation, then usually accurate and sparse representations are obtained when both the DPSS and the signal under investigation occupy the same band. However, problems arise when the signal is centered around some frequency |ωₒ|>0 and occupies bandwidth smaller than 2W. In such situations, a larger number of DPSS is required to approximate the signal with the same accuracy despite the fact that narrowband signals are more predictable than wider band signals. In order to find a better basis, MDPSS have been proposed. MDPSS are defined as:

Mₖ(N,W,ωₘ;n)=exp(jωₘn)vₖ(N,W;n),

where ωₘ=2πfₘ is a modulating frequency. It is easy to see that MDPSS are also doubly orthogonal, obey the same equation:

$${\sum }_{m = 0}^{N - 1} \frac{sin [ 2 π W ( n - m ) ]}{π ( n - m )} {v}_{k} ( m , N , W ) = {λ}_{k} ( N , W ) {v}_{k} ( n , N , W ) k = 0 , 1 , … , N - 1 ,$$

and are bandlimited to the frequency band [−W+ωₘ:W+ωₘ].

The next question which needs to be answered is how to choose a proper modulation frequency ωₘ. In the simplest case when the spectrum S(ω) of the signal is confined to a known band [ω₁; ω₂], i.e.,

$$S ( ω ) = \{ \begin{matrix}≫ 0 & ∀ ω ∈ [ {ω}_{1} , {ω}_{2} ] and \vert {ω}_{1} \vert < \vert {ω}_{2} \vert \\ \approx 0 & elsewhere\end{matrix} ,$$

then the modulating frequency, ωₘ, and the bandwidth of the DPSSs are naturally defined by

$${ω}_{m} = \frac{{ω}_{1} + {ω}_{2}}{2}$$

$$W = \vert \frac{{ω}_{2} - {ω}_{1}}{2} \vert ,$$

as long as both satisfy:

|ωₘ═+W<½.

However, in practical applications, the exact frequency band is only known with a certain degree of accuracy and usually evolves in time. Therefore, only some relatively wide frequency band is expected to be known. In such situations, an approach based on one-band-fits-all may not produce a sparse and accurate approximation of the signal. In order to resolve this problem, it has been suggested to use a band of bases with different widths to account for time-varying bandwidths. However, such a representation once again ignores the fact that the actual signal bandwidth could be much less than 2W dictated by the bandwidth of the DPSS. In order to provide further robustness to the estimation problem, the present invention employs a time-frequency dictionary containing bases which reflect various bandwidth scenarios.

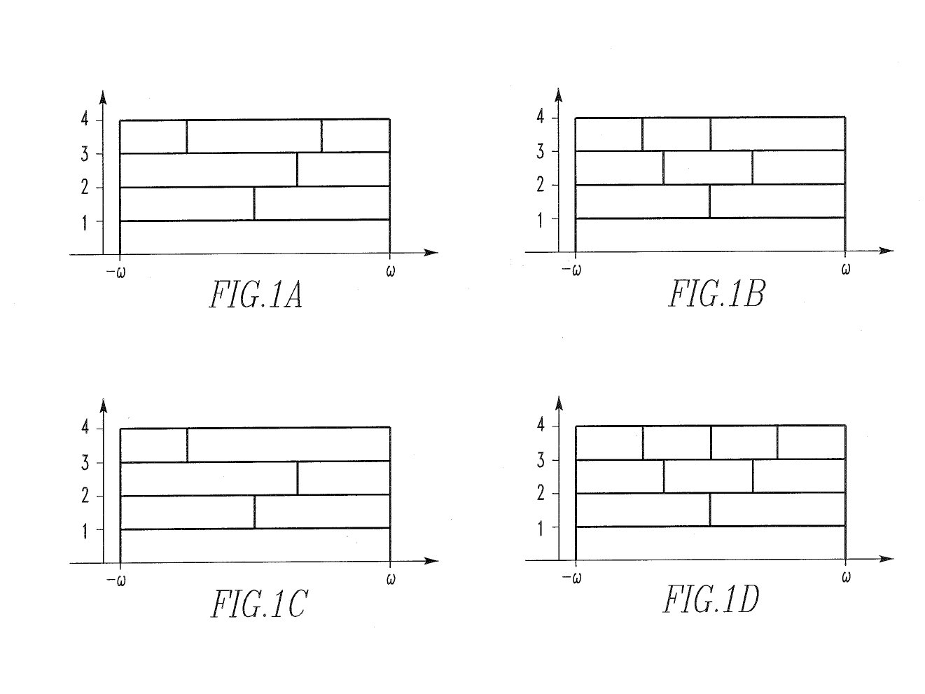

To construct this time-frequency dictionary, it is assumed that an estimate of the maximum frequency is available. The first few bases in the dictionary are the actual traditional DPSS with bandwidth W. Additional bases could be constructed by partitioning the band [−ω; ω] into K sub-bands with the boundaries of each sub-band given by [ωₖ; ωₖ₊₁], where 0≦k≦K−1, ωₖ₊₁>ωₖ, and ω₀=−ω, ω_{K-1}=ω. Hence, each set of MDPSS has a bandwidth equal to ωₖ₊₁−ωₖ and a modulation frequency equal to ωₘ=0.5(ωk+ωₖ₊₁). A set of such function again forms a basis of functions limited to the bandwidth [−ω; ω]. While the particular partition is arbitrary for every level K≧1, the bandwidth may be partitioned in any desired way such as is shown in FIGS. 1A-1D. In the exemplary embodiment, the bandwidth is partitioned in equal blocks as shown in FIG. 1D to reduce amount of stored pre-computed DPSS. In general, finding the best partitioning approach would be based on a priori knowledge about the phenomenon under investigation. Unless such knowledge is available, there is no strong reason to believe that non-uniform approaches shown in FIG. 1A-1C would yield a better performance than the uniform partitioning scheme shown in FIG. 1D without extensive optimization procedures.

As stated elsewhere herein, the CS approaches can be NP-hard, which are not practically viable. Fortunately, efficient algorithms, known generically in the art as matching pursuit algorithms, can be used to avoid some of the computational burden associated with the CS. A main feature of a matching pursuit algorithm is that, when stopped after a few steps, it yields an approximation using only a few basis functions. The matching pursuit decomposes any signal into a linear expansion of waveforms that are selected from a redundant dictionary of functions. It is a general, greedy, sparse function approximation scheme with the squared error loss, which iteratively adds new functions (i.e. basis functions) to the linear expansion. In comparison to a basis pursuit, it significantly reduces the computational complexity, since the basis pursuit minimizes a global cost function over all bases present in the dictionary. If the dictionary is orthogonal, the method works perfectly. Also, to achieve compact representation of the signal, it is necessary that the atoms are representative of the signal behavior and that the appropriate atoms from the dictionary are chosen.

In the exemplary embodiment, the algorithm for the matching pursuit starts with initial approximation for the signal, \hat{x} and the residual, R:

\hat{x}⁽⁰⁾(m)=0

R⁽⁰⁾(m)=χ(m),

where m represent the M time indices that are uniformly or non-uniformly distributed. Then, the matching pursuit builds up a sequence of sparse approximation by reducing the norm of the residue, R=\hat{x}−x. At stage k, it identifies the dictionary atom that best correlates with the residual and then adds to the current approximation a scalar multiple of that atom, such that:

\hat{x}^{(k)}(m)=\hat{x}^{(k-1)}(m)+αₖφₖ(m)

R^{(k)}(m)=x(m)−\hat{x}^{(k)}(m),

where αₖ=(R^{(k-1)}(m),φₖ(m))/∥φₖ(m)∥². The process continues till the norm of the residual R^{(k)}(m) does not exceed required margin of error ε>0: ∥R^{(k)}(m)∥≦ε.

In the exemplary embodiment, two stopping approaches are considered. One is based on the idea that the normalized mean square error should be below a certain threshold value, γ:

$$\frac{{‖ x - {\hat{x}}^{( k )} ‖}_{2}^{2}}{{‖ x ‖}_{2}^{2}} \leq γ .$$

An alternative stopping rule can mandate that the number of bases, n₂₃, needed for signal approximation should satisfy n₂₃≦κ. κ is set equal to [2/VW]+1 to compare the performance of the MDPSS-based frames with DPSS.

In either case, a matching pursuit approximates the signal using L bases as

$$x ( n ) = {\sum }_{l = 1}^{L} ( x ( m ) , {φ}_{l} ( m ) ) {φ}_{l} ( n ) + {R}^{( L )} ( n ) ,$$

where φₗ are L bases from the dictionary with the strongest contributions.

Based on the definition of MDPSS, it is desirable to know when the sampling times occur in order to use a proper value of the basis function. However, this is typically not realized and instead it is necessary to estimate the time location. Therefore, if it is assumed that the signal

$$x ( t ) = {\sum }_{m = 0}^{M - 1} x ( {\hat{t}}_{m} ) δ ( t - {\hat{t}}_{m} ) + n ( t )$$

is a superposition of M delta functions with additive noise n(t) resulting from the non-uniform sampling. To estimate \hat{t}ₘ, first consider the period extension of the signal:

$$x ( t ) = {\sum }_{k = - ∞}^{∞} {X}_{ic} {e}^{j k {Ω}_{o} t} + n ( t ) ,$$

where Ωₒ=2π/T and the Fourier coefficients are given by:

$${X}_{k} = {\sum }_{m = 0}^{M - 1} x ( {\hat{t}}_{m} ) {e}^{- j k {Ω}_{o} {\hat{t}}_{m}} = {\sum }_{m = 0}^{M - 1} x ( {\hat{t}}_{m} ) {u}_{m}^{k} - ( M - 1 ) \leq k \leq ( M - 1 ) ,$$

where uₘ=e^{-jΩ}^{ₒ}^{\hat{t}}^{ₘ}. The problem is then to find the parameters \hat{t}ₘ that satisfy the above equation from the noisy non-uniform samples, which can be achieved using the well known annihilating filter. In particular, if the transfer function of the annihilating filter is defined as:

$$A ( z ) = {\prod }_{m = 0}^{M - 1} ( 1 - {u}_{m} {z}^{- 1} ) = {\sum }_{m = 0}^{M - 1} {α}_{m} {z}^{- m} ,$$

then by filtering both sides of the equation for Xₖ above using the filter, the following is obtained:

$${\sum }_{m = 0}^{M - 1} {α}_{m} {X}_{k - m} = {\sum }_{m = 0}^{M - 1} {\sum }_{n = 0}^{N - 1} x ( {\hat{t}}_{n} ) {u}_{n}^{k - m} {α}_{m} = {\sum }_{m = 0}^{M - 1} x ( {\hat{t}}_{n} ) [ {\sum }_{n = 0}^{N - 1} {u}_{n}^{- m} {α}_{m} ] {u}_{n}^{k} ,$$

where the last term is due to uₙ being a root of A(z). Then, A(z) can be obtained by solving the equation immediately above for {αₘ} (i.e., set the equation equal to zero and solve for filter coefficients). Using the roots of A(z), uₘ=e^{-jΩ}^{ₒ}^{\hat{t}}^{ₘ}^{/T}, the non-uniform sampling times are estimated by:

$${\hat{t}}_{m} = \frac{- T}{2 π j} log {u}_{m} m = U , … , M - 1$$

A thorough description of the procedure can be found in Appendices A and B of M. Vetterli et al, “Sampling signals with finite rate of innovation,” IEEE Transactions on Signal Processing, vol. 50, no. 6, pp. 1417-1428, June 2002.

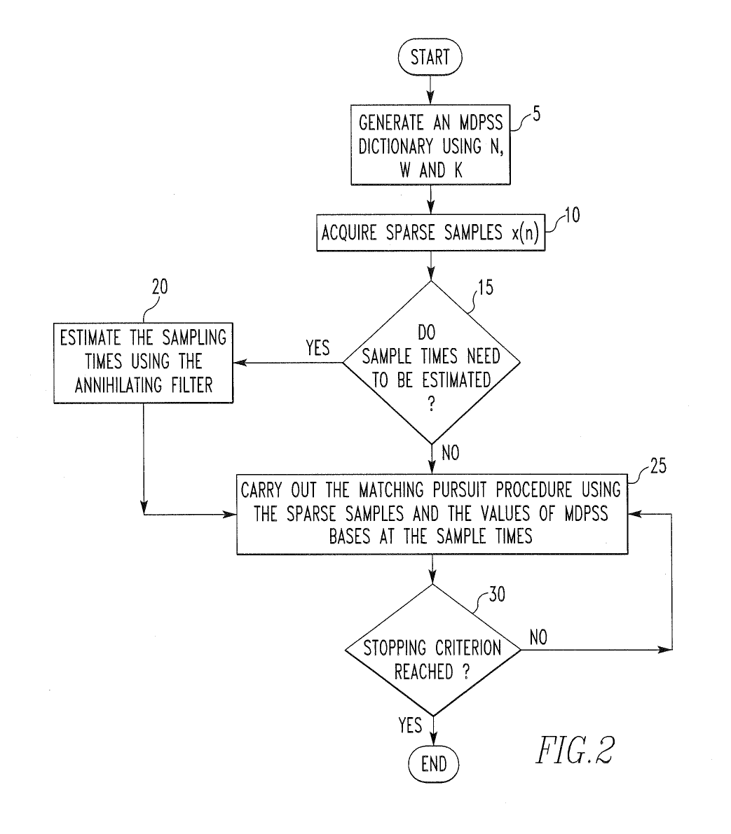

FIG. 2 is a flowchart illustrating a method of sampling and reconstructing an original physiological signal obtained from a subject according to the exemplary embodiment of the present invention. The method begins at step 5, wherein a time-frequency dictionary having bases which are modulated discrete prolate spheroidal sequences is generated using N, W and K, wherein N is the number of samples of the original signal that will be obtained, wherein the modulated discrete prolate spheroidal sequences are based on discrete prolate spheroidal sequences having a bandwidth W, and wherein K represents the number of bands (i.e., sub-bands) in the bandwidth of the discrete prolate spheroidal sequences. Next, at step 10, N samples of the original physiological signal are acquired. The, at step 15, a determination is made as to whether the sampling times of the acquired samples needs to be estimated. If the answer at step 15 is yes, then, at step 20, the sampling times are estimated using the annihilating filter as described herein. If the answer at step 15 is no, or after step 20, as the case may be, the method proceeds to step 25. At step 25, the matching pursuit algorithm is carried out using the acquired sparse signals (step 10) and the values of the MDPSS bases (step 5) at the sampling times. Next, at step 30, a determination is made as to whether a stopping criterion, as described herein, has been reached. If the answer is no, then the method returns to step 25. If the answer is yes, then the method ends as it is now possible to generate a reconstructed physiological signal. In the exemplary embodiment, the reconstructed physiological signal is output using a device such as a display and/or a printer.

FIG. 3 is a block diagram of a system 35 for day-to-day monitoring of swallowing disorders in which the method of the present invention (e.g., FIG. 2) may be implemented according to one particular, non-limiting exemplary embodiment. System 35 includes a dual-axis accelerometer 40 (e.g., the ADXL322 sold by Analog Devices) which is structured to be attached to a subject's neck (e.g., anterior to the cricoid cartilage) using a suitable connection method such as, without limitation, double-sided tape. In the exemplary embodiment, dual-axis accelerometer 40 is attached such that the axes of acceleration are aligned to the anterior-posterior and superior-inferior directions. System 35 also includes a band-pass filter 45 which receives the output of dual-axis accelerometer 40, and a computing device (described below) coupled to the output of filter 45. In such a configuration, data generated by dual-axis accelerometer 40 is band-pass filtered by filter 45 (e.g., with a pass band of 0.1-3000 Hz in the exemplary embodiment), and the filtered data is then sampled (e.g., without limitation, at 10 kHz) by computing device 50 (e.g., using a custom LabVIEW program running on computing device 50).

Computing device 50 may be, for example and without limitation, a PC, a laptop computer, a tablet computer, a smartphone, or any other suitable device structured to perform the functionality described herein. Computing device 50 is structured and configured to receive the filtered data output by filter 45 and process the data using an embodiment of the method described in detail herein in order to sample and reconstruct the original physiological swallowing signal obtained from the subject.

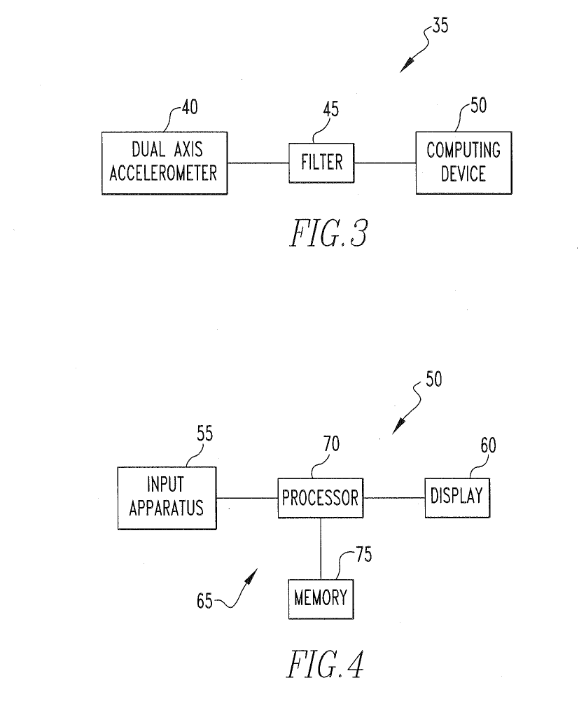

FIG. 4 is a block diagram of computing device 50 according to one exemplary embodiment. As seen in FIG. 4, the exemplary computing device 50 is a PC or laptop computer and includes an input apparatus 55 (which in the illustrated embodiment is a keyboard), a display 60 (which in the illustrated embodiment is an LCD), and a processor apparatus 65. A user is able to provide input into processor apparatus 65 using input apparatus 55, and processor apparatus 65 provides output signals to display 60 to enable display 60 to display information to the user, such as, without limitation, a reconstructed physiological signal generated using the method of the present invention. Processor apparatus 65 comprises a processor 70 and a memory 75. Processor 70 may be, for example and without limitation, a microprocessor (μP), a microcontroller, or some other suitable processing device, that interfaces with memory 75. Memory 75 can be any one or more of a variety of types of internal and/or external storage media such as, without limitation, RAM, ROM, EPROM(s), EEPROM(s), FLASH, and the like that provide a storage register, i.e., a machine readable medium, for data storage such as in the fashion of an internal storage area of a computer, and can be volatile memory or nonvolatile memory. Memory 75 has stored therein a number of routines that are executable by processor 70. One or more of the routines implement (by way of computer/processor executable instructions) at least one embodiment of the method discussed in detail herein for sampling and reconstructing a physiological signal using a time-frequency dictionary based on modulated discrete prolate spheroidal sequences.

In order to assess the performance of the method of the present invention, and in particular the method of FIG. 2 and the system 35 of FIG. 3, the present inventors performed a two part data analysis. In the first part, the present inventors considered synthetic test signals in order to examine the accuracy of the scheme in well-known conditions. In the second part, the present inventors used dual-axis swallowing accelerometry signals (FIG. 3) to examine how accurately these signals can be recovered from sparse samples. In both cases, the present inventors followed the method shown in FIG. 2.

### Part One—Synthetic Test Signals

To analyze the scheme of the present invention, the present inventors assumed the following test signal:

$$x ( n ) = {\sum }_{i = 1}^{10} {A}_{i} sin ( 2 π {f}_{i} n {T}_{s} ) + σ ζ ( n )$$

where 0≦n<N, Tₛ=1/256, N=256, Aᵢ is uniformly drawn from random values in [0, 2] and fᵢ˜N(30, 10²). ζ(n) represents white Gaussian noise and σ is its standard deviation.

A first experiment consisted of maintaining 150 samples equally spaced throughout the signal. The SNR values were varied between 0 dB and 30 dB in 1-dB increments, while the normalized half-bandwidth W was altered between 0.300 and 0.375 in 0.025 increments. The present inventors compared the accuracy of the approach of the present invention using 7-band and 15-band MDPSS-based dictionaries against the CS approach based on DPSS. The accuracy was compared by evaluating the normalized mean square error:

$$MSE = \frac{{‖ x ( n ) - \hat{x} ( n ) ‖}_{2}^{2}}{{‖ x ( n ) ‖}_{2}^{2}} ,$$

where x (n) is a realization of the signal defined by the assumed test signal equation above and \hat{x}(n) represents a recovered signal. The MSE values were obtained using 1000 realizations. To calculate the recovered signal using the DPSS, the present inventors used the following formula

\hat{x}_{DPSS}(n)=U(n,k)(U(m,k)^{T}U(m,k))^{†}U(m,k)^{T}x(m),

where A† denotes the pseudo-inverse of a matrix; U (n, k) is the matrix containing K bases (i.e., DPSS) and each sequence is of length N; m denotes the time instances at which the samples are available.

In a second experiment, the present inventors varied the number of available samples from 50 samples to 200 samples in increments of 10 samples in order to understand how the number of samples affects the overall accuracy of the scheme of the present invention. The samples were uniformly distributed, and the normalized half-bandwidth was set to 0.30. The lower boundary of 50 samples denotes a very aggressive scheme, as it represents approximately 20% of the original samples. On the other hand, the upper boundary of 200 samples represents a very lenient scheme for compressive sampling since it represents approximately 78% of the original samples. Additionally, the following four SNR values were used: 5 dB, 15 dB, 25 dB and 35 dB. The accuracy of CS-approach of the present invention was examined using 7-band and 15-band MDPSS based dictionaries against the CS-approach based on DPSS. The accuracy metric was the MSE value defined above, and 1000 realizations were used to obtain its values.

A third experiment examined the effects of non-uniform sampling times on the overall performance of the CS-based schemes. In particular, the present inventors used 100 non-uniform samples and the SNR values were incremented by 1 dB from 0 dB to 30 dB. Also, the normalized half-bandwidth was varied in 0.025 increments from 0.30 to 0.375. The accuracy of the approach of the present invention based on MDPSS was compared against the CS-approach based on DPSS. Specifically, the present inventors use 7-band and 15-band MDPSS-based time-frequency dictionaries. The accuracy metric was again the MSE value defined above. 1000 realizations were used again to obtain the MSE values, and for each realization new 100 time positions were achieved.

### Part Two—Swallowing Accelerometry Signals

Using the scheme of the present invention, the present inventors analyzed how accurately dual-axis swallowing accelerometry signals can be recovers from sparse samples. Specifically, the present assumed two different scenarios: (i) only 30% of the original samples are available, and (ii) only 50% of the original samples are available. In both cases, the present inventors examined whether the uniform or non-uniform sub-Nyquist rates have significant effects on the overall effectiveness of the scheme. In this numerical experiment, the present inventors used a 10-band MDPSS based dictionary with the normalized half-bandwidth equal to 0.15. To evaluate the effectiveness of the approach when considering dual-axis swallowing accelerometry signals, the present inventors adopted performance metrics used in other biomedical applications.

Those metrics include Cross-correlation (CC), Percent root difference (PRD), Root mean square error (RMSE), and Maximum error (MAXERR). CC is used to evaluate the similarity between the original and the reconstructed signal, and is defined as:

$$CC = \frac{{\sum }_{n = 1}^{N} ( x ( n ) - {μ}_{x} ) ( \tilde{x} ( n ) - {μ}_{\tilde{x}} )}{\sqrt{{\sum }_{n = 1}^{N} {( x ( n ) - {μ}_{x} )}^{2}} \sqrt{{\sum }_{n = 1}^{N} {( \tilde{x} ( n ) - {μ}_{\tilde{x}} )}^{2}}} \times 100 \% ,$$

where x(n) is the original signal and \hat{x}(n) represents a reconstructed signal. In addition, μₓ and μ_{\hat{x}} denote the mean values of x(n) and \hat{x}(n), respectively. PRD measures distortion in reconstructed biomedical signals, and is defined as:

$$PRD ( \% ) = \sqrt{\frac{{\sum }_{n = 1}^{N} {( x ( n ) - \hat{x} ( n ) )}^{2}}{{\sum }_{n = 1}^{N} {x}^{2} ( n )}} \times 100 \% .$$

RMSE also measures distortion and is often beneficial to minimize this metric when finding the optimal approximation of the signal. RMSE is defined as:

$$RMSE = \sqrt{\frac{{\sum }_{n = 1}^{N} {( x ( n ) - \hat{x} ( n ) )}^{2}}{N}} .$$

MAXERR is used to understand the local distortions in the reconstructed signal, and it particularly denotes the largest error between the samples of the original signal and the reconstructed signal, and is defined as:

MAXERR=max(x(n)−\hat{x}(n)).

In order to establish statistical significance of the results, a non-parametric inferential statistical method known as the Mann-Whitney test was used, which assesses whether observed samples are drawn from a single population (i.e., the null hypothesis). For multi-group testing, the extension of the Mann-Whitney test known as the Kruskal-Wallis was used. A 5% significance was used.

### Results

The results of the numerical experiments described above will now be discussed.

First, the results based on the synthetic test signals are discussed. Thereafter, the results of the numerical experiments considering the application of the approach of the present invention to dual-axis swallowing accelerometry signals are discussed.

Synthetic Test Signals

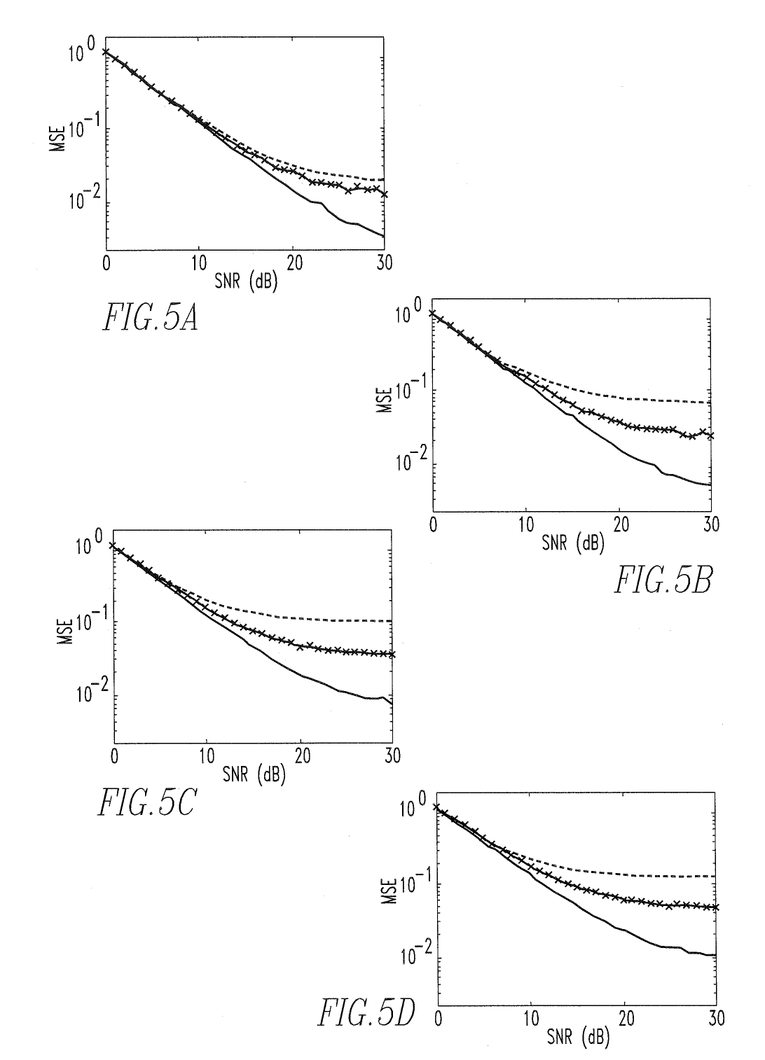

The results of the first numerical experiment are shown in FIGS. 5A-5D (the effects of increasing initial bandwidth of discrete prolate sequences), wherein in FIG. 5A, W=0.300, in FIG. 5B, W=0.325, in FIG. 5C, W=0.350, and in FIG. 5D, W=0.375, wherein the dashed lines denote MSE obtained with the DPSS, the solid lines indicate MSE obtained with a 15-band MDPSS-based dictionary, and the solid line with Xs denote a 7-band MDPSS-based dictionary. Several observations are in order. First, the approach for CS based on the time-frequency dictionary containing MDPSS achieved more accurate signal reconstructions than the CS approach based on DPSS. This can be observed regardless of the initial bandwidth used for discrete prolate sequences. Second, the CS approaches based on both MDPSS and DPSS bases provide similar accuracy at very low SNR values (e.g., SNR<5 dB).

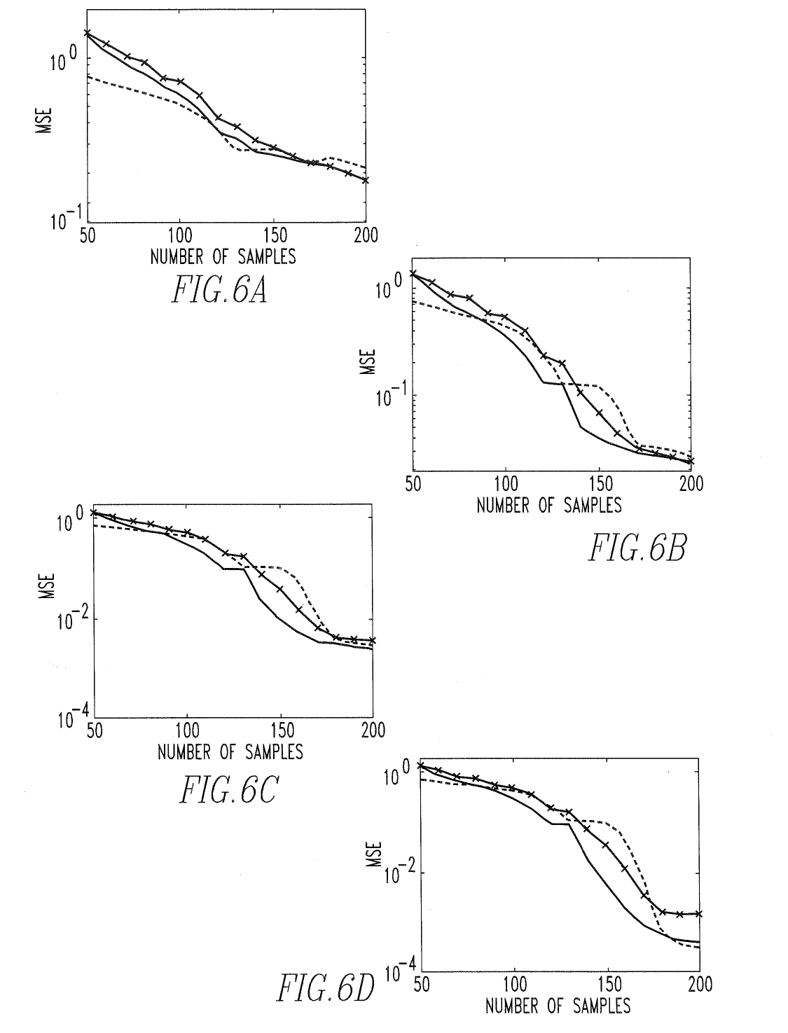

The results of the second numerical experiment are shown in FIGS. 6A-6D (increasing number of samples used in CS while altering the SNR values), wherein in FIG. 6A, SNR=5 dB, in FIG. 6B, SNR=15 dB, FIG. 6C, SNR=25 dB, and FIG. 6D, SNR=35 dB, and wherein the dashed lines denote MSE obtained with the DPSS, the solid line indicates MSE obtained with a 15-band MDPSS-based dictionary, and the solid lines with Xs denote a 7-band MDPSS-based dictionary. As expected, CS approaches based on MDPSS and DPSS have similar accuracies for a low SNR value (i.e., SNR=5 dB) as shown in FIG. 6A. Both types of bases (i.e., MDPSS and DPSS) are not suitable for accurate representations of random variables, and possibly dictionaries based on random bases would be a more suitable approach for low SNR values. As SNR increases, the MSE decreases for both approaches and the CS approach based on MDPSS obtains higher accuracy. The results also showed that if the percent of available samples is below 30 (i.e., acquiring signals at rates that are 30% of the original Nyquist rate), the DPSS and MDPSS based schemes achieve similar accuracy.

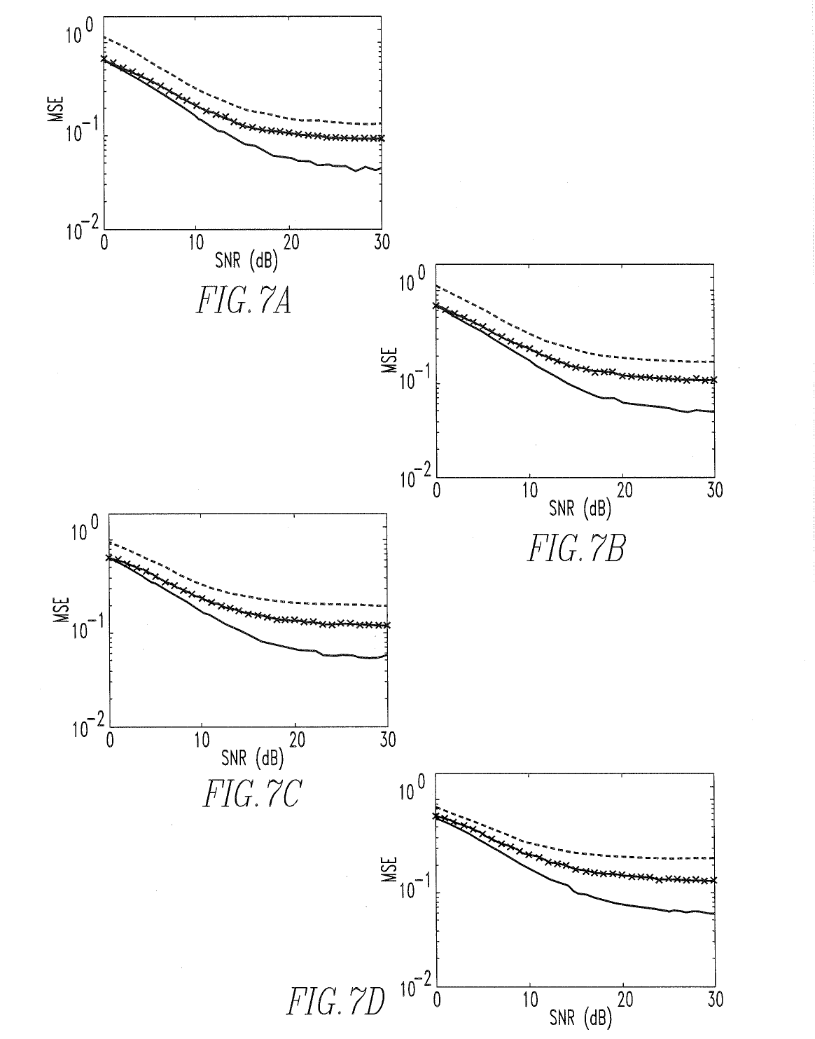

The results of third numerical experiment are summarized in FIGS. 7A-7D (the effects of random time positions of samples on the accuracy of the scheme while altering the bandwidth of discrete prolate sequences), wherein in FIG. 7A, W=0.300, in FIG. 7B, W=0.325, in FIG. 7C, W=0.350, and in FIG. 7D, W=0.375, and wherein the dashed lines denote MSE obtained with the DPSS, the solid lines indicate MSE obtained with a 15-band MDPSS-based dictionary, and the solid lines with Xs denote a 7-band MDPSS-based dictionary. FIGS. 7A-7D clearly depict the advantage of the CS approach based on the MDPSS over the approach based on DPSS even if non-uniform sampling is used. For all four considered cases, more accurate results were achieved with MDPSS than with DPSS. Additionally, more accurate results were achieved when the 15-band dictionary was used rather than the 7-band dictionary. This is in accordance with the previous results shown in FIGS. 5A-5D, which also showed that more comprehensive dictionaries can provide more accurate results due to the fact that they can account for many different time-varying bandwidth scenarios. CS of swallowing accelerometry signals

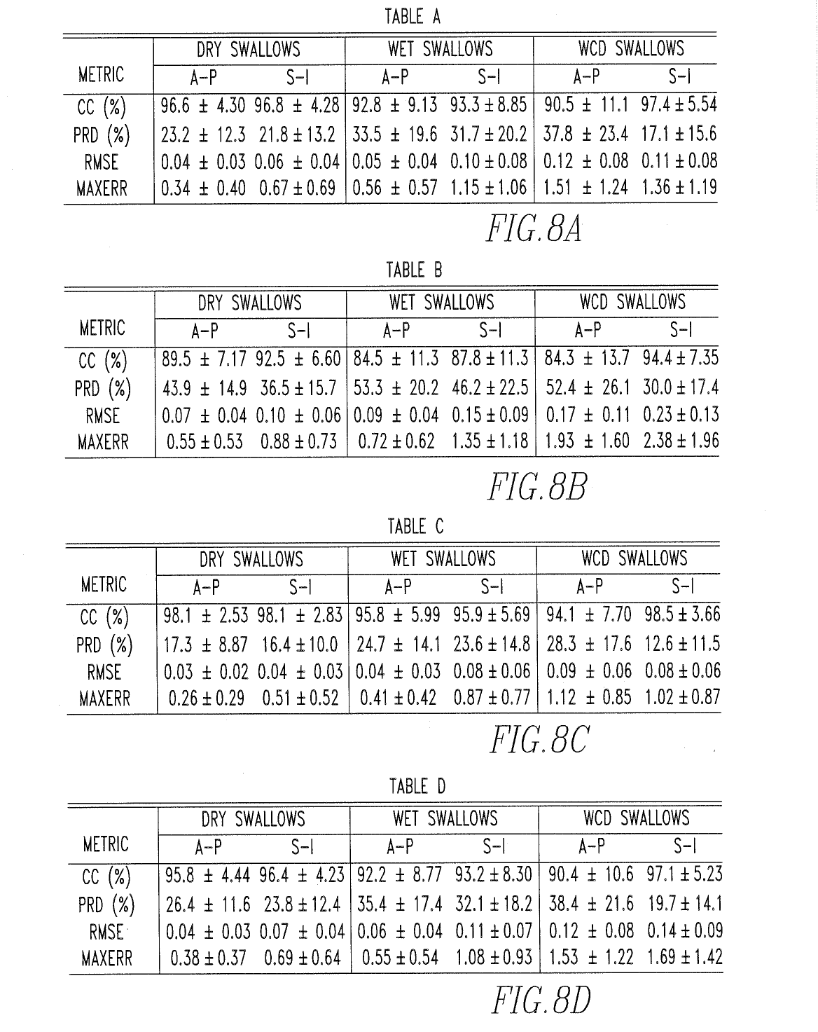

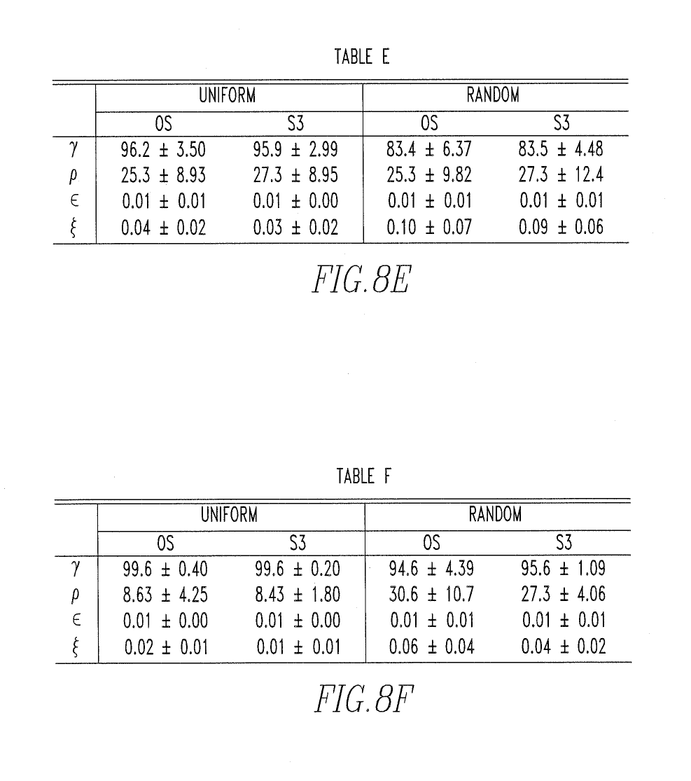

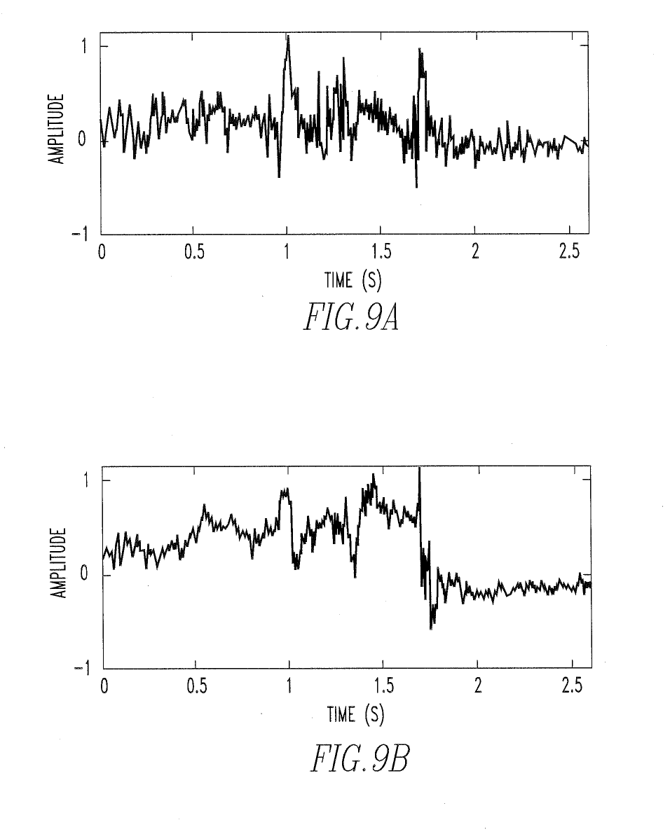

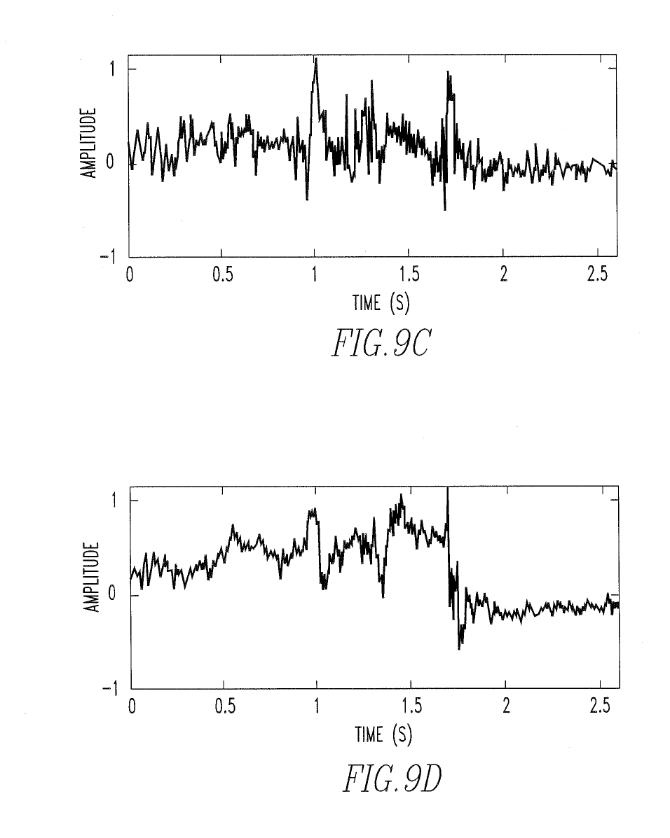

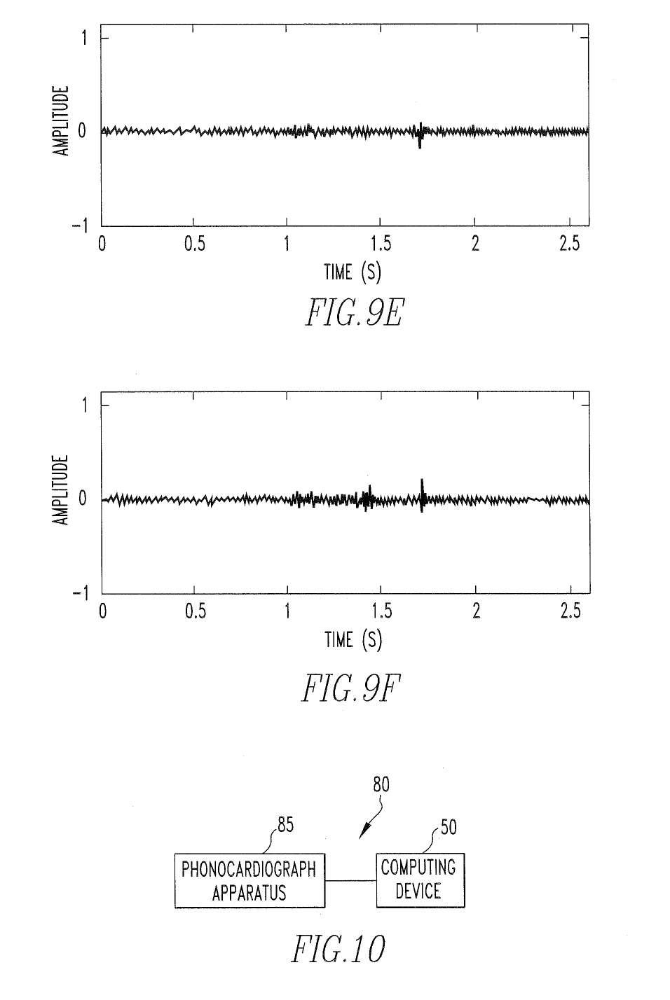

Tables A-D, shown in FIGS. 8A-8D, respectively, depict the results of the numerical analysis when the scheme of the present invention is applied to dual-axis swallowing accelerometry signals. Sample signals are shown in FIGS. 9A-9F, wherein FIG. 9A shows the original signal in the A-P direction, FIG. 9B shows the original signal in the S-I direction, FIG. 9C shows the recovered signal in the A-P direction (50% samples, CC=99.7%), FIG. 9D shows the recovered signal in the S-I direction (50% samples, CC=99.8%), FIG. 9E shows the error between the original and the recovered signal in the A-P direction, and FIG. 9F shows the error between the original and the recovered signal in the S-I direction. Several observations are in order.

First, a very high agreement was achieved between the reconstructed data and the original signals with uniformly spread out samples. Statistically higher results were achieved with 50% of samples as compared to 30% of samples when considering the cross-correlations results (p<<0.01), which resulted in statistically lower errors with 50% of samples when considering the three error metrics (p<<0.01).

Second, statistically worse results were obtained when using non-uniform (random) sampling times (p<<0.01) in comparison to uniform sampling for both 30% of samples and 50% of samples. This result is expected, as it becomes more challenging to recover the signal accurately with non-uniform samples. Additionally, it is difficult to recover swallowing vibrations accurately, given that these vibrations are short-duration transients. Unless the non-uniform samples capture the behavior of these short-duration transients, a larger recovery error is achieved. However, with 50% of samples, very high agreement was obtained between the recovered data and the original signals. As a matter of fact, the results obtained with 50% of samples with non-uniform sampling are comparable to the results obtained with 30% of samples when using uniform sampling. Third, amongst the considered swallowing tasks, dry swallows tend to be recovered most accurately, followed by the wet swallows and lastly by the wet chin down swallows. From a physiological point of view, this is expected since during the dry swallowing maneuver, only small amounts of liquid (i.e., saliva) are swallowed. It is also expected that wet chin down swallows will be more difficult to recover due to the complex maneuvering required during these swallows, which may introduce signal components otherwise not present during the dry and/or wet swallowing tasks.

Therefore, based on the presented results, it can be stated with a high confidence that CS based on the time-frequency dictionary containing MDPSS of the present invention is a suitable scheme for dual-axis swallowing accelerometry signals. Particularly accurate results have been obtained 50% of samples are used.

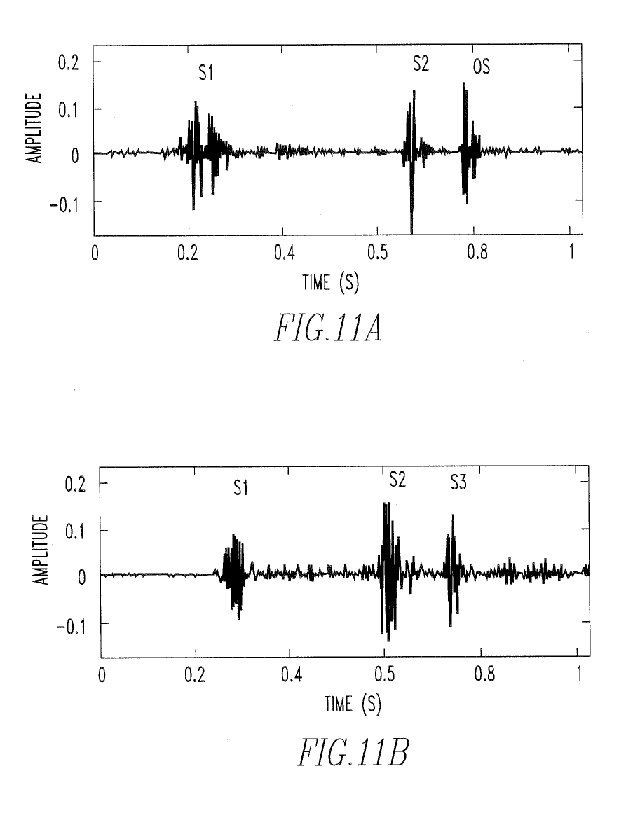

FIG. 10 is a block diagram of a system 80 for monitoring of heart sounds in which the method of the present invention (e.g., FIG. 2) may be implemented according to another particular, non-limiting exemplary embodiment. System 80 includes a phonocardiograph apparatus 80 which is coupled to computing device 50 as described elsewhere herein. Phonocardiograph apparatus 80 is a device that includes a number of microphones and recording equipment (e.g., an analog recorder/player such as the Cambridge AVR-I) that is structured to monitor and record heart sounds of a subject. In such a configuration, heart sound data generated by is sampled (e.g., without limitation, at 4000 Hz) by computing device 50 (e.g., using a custom LabVIEW program running on computing device 50). In the present embodiment, computing device 50 has stored therein a number of routines that implement (by way of computer/processor executable instructions) at least one embodiment of the method discussed in detail herein for sampling and reconstructing a signal representing heart sounds of a subject. In particular, system 80 is configured for recovering sparsely sampled heart sounds including recordings containing opening snap (OS) or the third heart sounds (S3) in addition to first and second heart sounds. As described below, the results of numerical analysis performed by the present inventors show that heart sounds can be accurately reconstructed even when the sampling rate is reduced to 40% of the original sampling frequency.

To examine the suitability of compressive sensing for heart sounds, the present inventors considered how accurately heart sounds can be recovered from sparse samples using the method of the present invention. To examine the recovery accuracy, the present inventors considered two different scenarios where a different number of samples were available. First, the present inventors considered when 40% of the original samples were available, and second, the present inventors considered when 60% of the original samples were available. For both cases, the effects of the uniform or non-uniform sub-Nyquist sampling were examined. In this numerical experiment, the present inventors used a 10-band MDPSS based dictionary with the normalized half bandwidth equal to 0.25. The effectiveness of compressive sensing of heart sounds was evaluated through performance metrics used in other biomedical applications as described elsewhere herein.

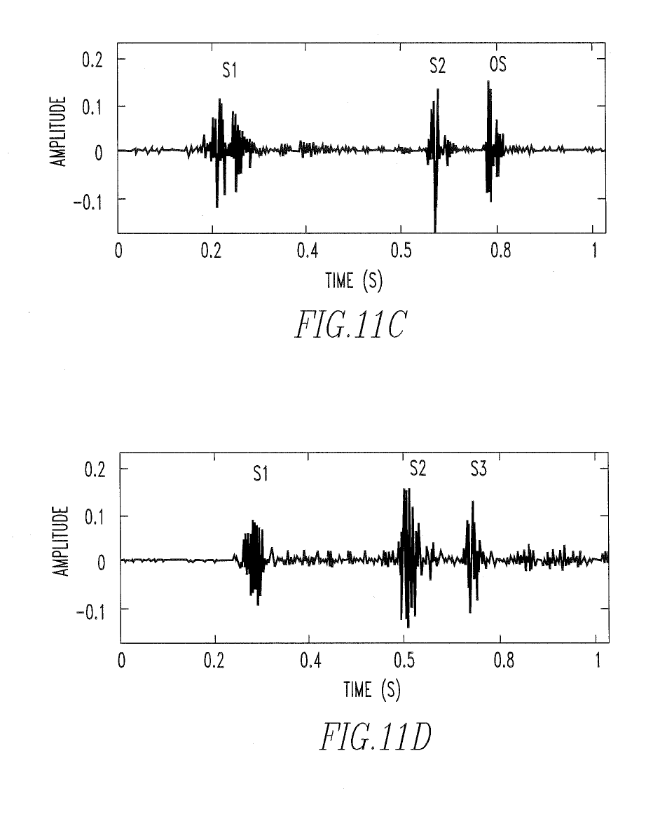

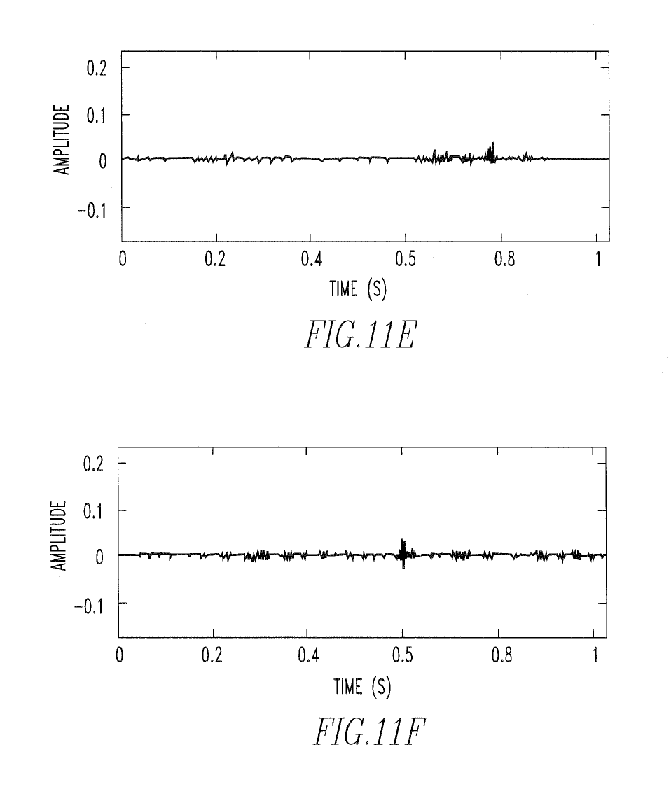

Sample signals from the experiment are shown in FIGS. 11A-11F, while Tables E and FR shown in FIGS. 8E and 8F, respectively, show the results of the numerical analysis when the scheme of the present invention is applied to heart sounds. FIG. 11A shows the original signal containing the OS, FIG. 11B shows the original signal containing the S3, FIG. 11C shows the recovered signal containing the OS (40% samples, γ=98.9%), FIG. 11D shows the recovered signal containing the S3 (40% samples, γ=99.0%), FIG. 11E shows the error between the original and the recovered signal with the OS, and FIG. 11F shows the error between the original and the recovered signal with the OS.

The results show that the heart sounds can be very accurately reconstructed from the sparsely sampled recordings. As expected, more accurate results were achieved with 60% of samples than with 40% of samples when considering the cross-correlations (γ) results. Less accurate results were obtained when using non-uniform (random) sampling times in comparison to uniform sampling for both 40% of samples and 60% of samples. These results follow the previously reported trends which show that it is more challenging to recover the signal accurately with non-uniform samples. However, when a larger number of samples (e.g., 60% of samples) is used, the original signals can be recovered very accurately from randomly sparse samples. Specifically, the results obtained with 60% of samples with non-uniform sampling are comparable to the results obtained with 40% of samples when using uniform sampling. The results also show that the considered CS approach is an accurate reconstruction method regardless of the present heart sounds. Specifically, the present inventors considered recordings containing OS or S3 in addition to first and second heart sounds. The presented results showed that CS approach based on MDPSS is robust to changes in the underlying physiological process, which is a desirable property from a systems point of view. This inherently implies that any future systems developed for sparse sampling of heart sounds can use a uniform sampling scheme regardless of the present physiological phenomena.

Therefore, based on the results, it can be stated with confidence that CS based on the time-frequency dictionary containing MDPSS is a suitable scheme for heart sounds. Particularly accurate results have been obtained when 60% of samples is used.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A method of sampling and reconstructing an original physiological signal obtained from a subject, comprising:
acquiring a number of samples of the original physiological signal; and
generating a reconstructed physiological signal using the samples and a time-frequency dictionary, the time-frequency dictionary having bases which are modulated discrete prolate spheroidal sequences.

2. The method according to claim 1, wherein the acquiring the samples comprises sampling the original physiological signal at a sample rate that is less than a Nyquist rate of the original physiological signal.

3. The method according to claim 1, wherein the time-frequency dictionary comprises a number of values, wherein the generating the reconstructed physiological signal comprises employing a matching pursuit algorithm using each of the samples and each of the values of the time-frequency dictionary.

4. The method according to claim 3, wherein each of the samples is associated with a respective sampling time, wherein each sample has one of the values of the time-frequency dictionary that corresponds thereto that is also associated with the respective sampling time of the sample, wherein matching pursuit algorithm is, for each of the samples, carried out using the one of the values of the time-frequency dictionary corresponding to the sample.

5. The method according to claim 4, wherein each of the sampling times is estimated.

6. The method according to claim 5, wherein each of the sampling times is estimated using an annihilating filter.

7. The method according to claim 1, wherein the generating the reconstructed physiological signal employing the matching pursuit algorithm further comprises determining that a stopping criterion has been reached and in response thereto outputting the reconstructed physiological signal.

8. The method according to claim 1, further comprising generating the time-frequency dictionary, wherein the number of samples is N, wherein the modulated discrete prolate spheroidal sequences are based on discrete prolate spheroidal sequences having a bandwidth W, wherein K represents a number of bands in the bandwidth of the discrete prolate spheroidal sequences, and wherein the time-frequency dictionary is generated based on N, W and K.

9. The method according to claim 1, further comprising outputting the reconstructed physiological signal.

10. The method according to claim 9, wherein the outputting the reconstructed physiological signal comprises displaying the reconstructed physiological signal on a display device.

11. The method according to claim 1, wherein the original physiological signal represents swallowing signals generated by the subject.

12. The method according to claim 11, wherein the acquiring the number of samples of the original physiological signal is performed using a dual axis accelerometer.

13. The method according to claim 1, wherein the original physiological signal represents heart sounds of the subject.

14. A computer program product, comprising a computer usable medium having a computer readable program code embodied therein, the computer readable program code being adapted to be executed to implement a method for sampling and reconstructing an original physiological signal obtained from a subject as recited in claim 1.

15. A system for sampling and reconstructing an original physiological signal obtained from a subject, comprising:
an output device; and
a computing device having a processor apparatus structured and configured to:
receive a number of samples of the original physiological signal;
generate a reconstructed physiological signal using the samples and a time-frequency dictionary, the time-frequency dictionary having bases which are modulated discrete prolate spheroidal sequences; and
cause the reconstructed physiological signal to be output on the output device.

16. The system according to claim 15, wherein the output device is a display.

17. The system according to claim 15, wherein the samples are obtained by sampling the original physiological signal at a sample rate that is less than a Nyquist rate of the original physiological signal.

18. The system according to claim 15, wherein the time-frequency dictionary comprises a number of values, wherein the reconstructed physiological signal is generated by employing a matching pursuit algorithm using each of the samples and each of the values of the time-frequency dictionary.

19. The system according to claim 18, wherein each of the samples is associated with a respective sampling time, wherein each sample has one of the values of the time-frequency dictionary that corresponds thereto that is also associated with the respective sampling time of the sample, wherein matching pursuit algorithm is, for each of the samples, carried out using the one of the values of the time-frequency dictionary corresponding to the sample.

20. The system according to claim 19, wherein processor apparatus structured and configured to estimate each of the sampling times.

21. The system according to claim 20, wherein the processor apparatus is structured and configured to estimate each of the sampling times using an annihilating filter.

22. The system according to claim 15, wherein the processor apparatus is structured and configured to determine that a stopping criterion has been reached and in response thereto output the reconstructed physiological signal.

23. The system according to claim 15, wherein the processor apparatus is structured and configured to generate the time-frequency dictionary, wherein the number of samples is N, wherein the modulated discrete prolate spheroidal sequences are based on discrete prolate spheroidal sequences having a bandwidth W, wherein K represents a number of bands in the bandwidth of the discrete prolate spheroidal sequences, and wherein the time-frequency dictionary is generated based on N, W and K.

24. The system according to claim 15, wherein the original physiological signal represents swallowing signals generated by the subject, and wherein the system further comprises an acoustic or vibration sensor for generating the original physiological signal.

25. The system according to claim 24, wherein the acoustic or vibration sensor is a dual axis accelerometer.

26. The system according to claim 15, wherein the physiological signal represents heart sounds of the subject, and wherein the system further comprises an acoustic sensor for generating the original physiological signal.

27. A system that facilitates monitoring of physiological function, comprising:
a sampling component that employs compressive sensing of biomedical signals associated with the physiological function; and
a dictionary component that employs time-frequency dictionaries based upon modulated discrete prolate spheroidal sequences (DPSS) to process the compressive sensing.

28. The system according to claim 27, wherein the physiological function includes swallowing.

29. The system according to claim 27, wherein the physiological function includes heart sounds.

30. The system according to claim 27, wherein the sampling component includes a compressive sensing (CS) algorithm that alleviates computational intensity while acquiring dual-axis swallowing accelerometry signals or heart sounds.

31. The system according to claim 30, further comprising a rendering component that generates and displays waveforms obtained by modulation and variation of DPSS in order to reflect the time-varying nature of the accelerometry signals.

32. The system according to claim 30, wherein a matching pursuit algorithm is adopted to iteratively decompose the signals into an expansion of the dictionary bases.

33. The system according to claim 27, wherein dual-axis swallowing accelerometry signals and/or heart sounds can be accurately reconstructed at a sampling rate reduced to half of a Nyquist rate of the biomedical signals associated with the physiological function.
